(19) United States
(12) Patent Application Publication
(10) Pub. No.: US 2017/0168323 A1
(43) Pub. Date: Jun. 15, 2017

(54) Method of Determining Eyeglass Fitting Measurements from an Image by Executing Computer-Executable Instructions Stored on a Non-Transitory Computer-Readable Medium

(71) Applicant: Kurt Matthew Gardner, Coral Springs, FL (US)

(72) Inventor: Kurt Matthew Gardner, Coral Springs, FL (US)

(21) Appl. No.: 15/443,880

(22) Filed: Feb. 27, 2017

Related U.S. Application Data
(63) Continuation-in-part of application No. 15/136,859, filed on Apr. 22, 2016.
(60) Provisional application No. 62/151,270, filed on Apr. 22, 2015.

(51) Int. Cl. G02C 13/00; G06T 3/40; G06T 7/62; G06K 9/00; A61B 3/11; A61B 3/00

(52) U.S. Cl. CPC G02C 13/005; A61B 3/111; A61B 3/0025; A61B 3/0058; G06T 7/62; G06K 9/00268; G06T 3/40; G06T 2207/30201

(57) ABSTRACT

A method of determining eyeglass fitting measurements from an image for a vision treatment analyzes a front facing source image of a subject to be fitted for frames. Anatomical features are measured from the source image. Attributes of the subject are used to correlate an expected true dimension of the anatomical features and thus scale the measurements in the source image to find the true measurements. More specifically, the subject's iris is measured in the source image, and the subject's height and weight are correlated with an expected iris size, thus enabling scaling of the measurements to find the true dimensions for supplying the subject with appropriately fitted eyeglass frames.

The current application is a continuation-in-part (CIP) application of a U.S. non-provisional application Ser. No. 15/136,859 filed on Apr.22, 2016. The U.S. non-provisional application Ser. No. 15/136,859 claims a priority to a U.S. provisional application Ser. No. 62/151,270 filed on Apr. 22, 2015.

### FIELD OF THE INVENTION

The present invention relates generally to eye care. More particularly, the present invention relates to a method for accurately determining eyeglass fitting measurements from a digital image.

### BACKGROUND OF THE INVENTION

In most cases an individual must go to an eye care professional to have measurements of their head taken to determine the correct eyeglass frame dimensions. Going to an eye care professional requires appointments to be made, which may not align with an individual's schedule. This method can prove to be time consuming and inconvenient for busy individuals and serves to standardize the skill set to take the measurements, which, when done manually, can lead to significant errors. Alternatively, there are methods of determining eyeglass fitting measurements using multiple images with attachments to the frame which currently exist in the market. Many times, methods such as this still involve the individual having to go in to see an eye care professional to get the proper pictures taken, leading to the same issue of time and convenience. Further, some methods do not require an individual to physically visit an eye care professional as they can take the required photos on their own and upload them onto the eye care provider's website. Although this method is quicker and more convenient than the aforementioned methods, an individual may not properly take the photos. This may result in inaccurate and incomplete results, having to retake the photos and overall may lead to more inconvenience and time wasted, and existing methods cannot complete all measurements required to make modern eyewear.

The present invention aims to solve the problems with determining eyeglass fitting measurements mentioned above. With the present invention, an individual will be able to take any front facing photo of themselves and determine accurate eyeglass fitting measurements for that individual. The present invention does not require the individual to me wearing a known pair of eyeglass frames, as anatomical measurements are used to properly scale the photo. Through scaling methods and trigonometry, an individual will not need to take multiple images (of the sides of the head, etc.) to accurately fit eye glass frames. Frame measurements can accurately be taken in the comfort of one's home, saving time and energy, as well as ensuring consistency that may not be adequately provided by an eye care professional.

### BRIEF DESCRIPTION OF THE DRAWINGS

FIG. 1 is a general overview of the process of the present invention.

FIG. 2 is an illustration showing several facial dimensions relevant to the present invention.

FIG. 3 is a stepwise flow diagram showing the general process of the present invention.

FIG. 4 is a stepwise flow diagram showing steps for determining the height and width scaling factors.

FIG. 5 is a stepwise flow diagram showing further steps for calculating the height and width scaling factors.

FIG. 6 is a stepwise flow diagram showing further steps for calculating the height and width scaling factors.

FIG. 7 is a stepwise flow diagram showing steps for calculating the true pupil distance.

FIG. 8 is a stepwise flow diagram showing steps for calculating the true segment height.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced without the implementation of some features as they are described. It should be understood that some details have not been described in detail in order to not unnecessarily obscure focus of the invention.

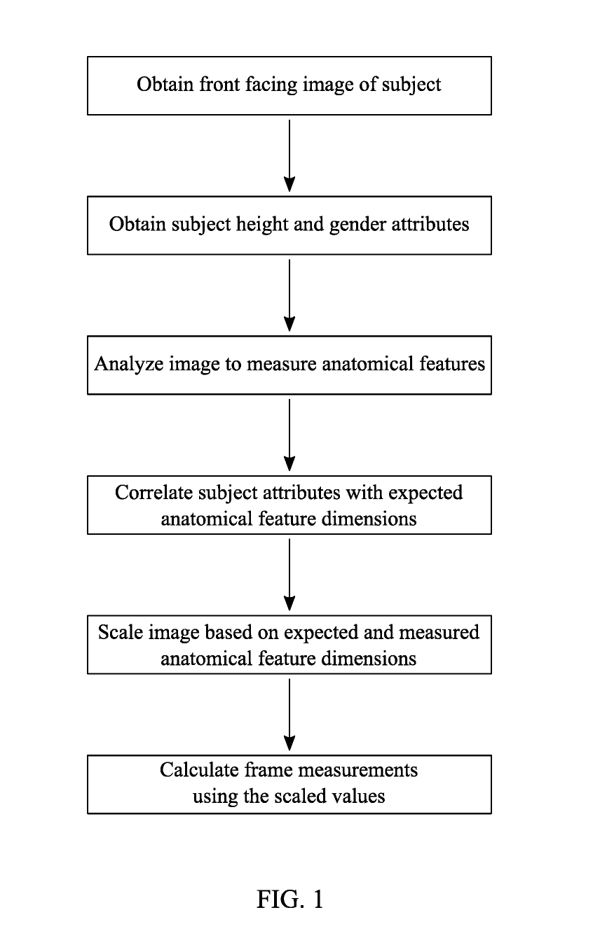

The present invention takes patient measurements for properly fitting eyeglass frames with the use of a single image of the wearer. By using facial recognition techniques and image scaling methods, the measurements specific to the patient can be accurately and consistently determined. The user will no longer have to take a plurality of specific photos to properly fit eyeglass frames using this method. In other words, one front facing photo will be all that is needed. The end goal of the present invention is to ascertain a pupil distance (PD) and segment height (SH) as the primary measurements for fitting eyeglass frames for a subject using only one or more anatomical features of the subject. FIG. 1 shows a general overview of the process of the present invention.

To facilitate the present invention, an image recognition software must be provided for use. The image recognition software may be any collection of programming code, compiled or otherwise, machine instructions, or any type of computer-executable instructions that provides the ability to analyze an image in order to recognize the required components from the image as detailed hereinafter. The image recognition software may be a single software package with all the necessary capabilities, or the image recognition software may be multiple software packages each providing one or more of said capabilities.

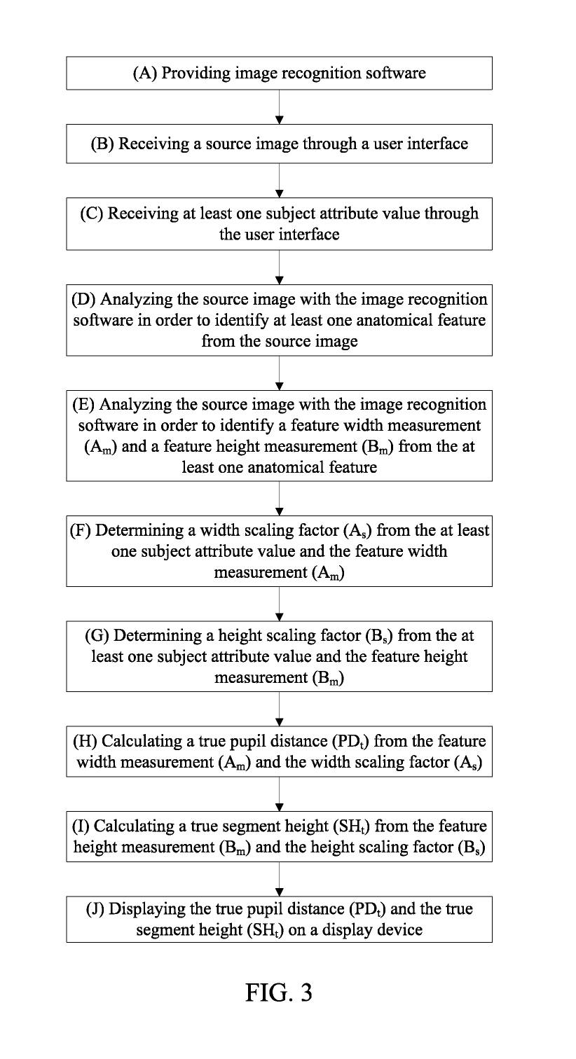

Referring to FIG. 3, to initiate the general process of the present invention, a source image is received through a user interface. The source image may be received from any applicable digital file transfer means and may be stored on and read from any digital file storage implementation. To properly enable the present invention, the source image must contain a front facing photo of a person's head. It is generally optional but preferable that the person also be wearing an existing eyeglass frame. The eyeglasses worn by the user should be recognizable enough to look up the manufacturers details on the dimensions. An existing photograph of an individual wearing a known pair of glasses will work with the present invention. For example, an individual may be able to use one of their photos from a social media website. In some embodiments it may be an additional feature of the present invention to utilize the image recognition software in order to automatically determine the model of the eyeglasses worn by the user in the source image.

At least one subject attribute value is furthermore received through the user interface. The key to the current invention is that there exists a strong correlation between a person's height and gender to the size of their iris. Therefore, by knowing the subject's height value and gender value, the source image can be analyzed and scaled with a reasonable degree of accuracy, thereby allowing accurate determination of the proper measurements to fit the subject for comfortable eyeglass frames. In the preferred embodiment of the present invention, both a subject height value and a subject gender value are received as the at least one subject attribute value.

The source image is then analyzed with the image recognition software in order to identify at least one anatomical feature from the source image. In the preferred embodiment, the at least one anatomical feature is the subject's iris. However, it is contemplated that in alternative embodiments other anatomical features may be utilized instead or in conjunction with the subject's iris. For example, measurements may be taken from the source image of the subject's nasal cavity, bridge width, distance between the nose and the top of the upper lip, distance between the top of the nose to the outer edge of the lip, or any other relevant features may be analyzed in addition to or instead of the iris which may be useful to the purpose of the present invention.

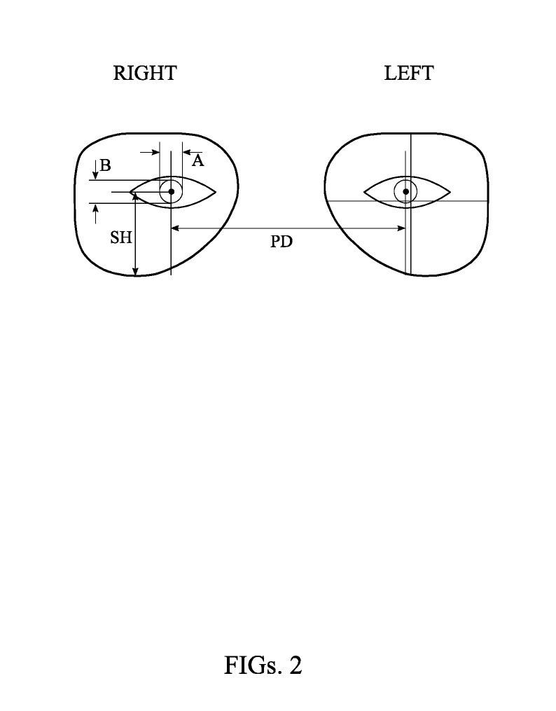

The source image is analyzed with the image recognition software in order to identify at least one feature dimension. An illustration of the relevant dimensions described herein is shown in FIG. 2. More specifically, in the preferred embodiment, a feature width measurement (Aₘ) and a feature height measurement (Bₘ) are identified from the at least one anatomical feature in the source image. Even more specifically, in the preferred embodiment the feature width measurement (Aₘ) and the feature height measurement (Bₘ) are the measured width and height of the subject's iris in the source image. Since in general the assumption can be made that a person's iris is circular, measuring both the height and width of the iris from the source image allows for correction for any deviance if the source image is not a completely straight-on image of the user's head. It should be understood that all variable nomenclature herein is arbitrary and is not intended in any way to limit the scope of the present invention.

After the source image is analyzed, scaling factors must be determined. A width scaling factor (Aₛ) is determined from the at least one subject attribute value and the feature width measurement (Aₘ), and a height scaling factor (Bₛ) is determined from the at least one subject attribute value and the feature height measurement (Bₘ). A true pupil distance (PDₜ) is calculated from the feature width measurement (Aₘ) and the width scaling factor (Aₛ), and a true segment height (SHₜ) is calculated from the feature height measurement (Bₘ) and the height scaling factor (Bₛ). The true pupil distance (PDₜ) and the true segment height (SHₑ) are then stored in a database and displayed on a display device to an end user so that the values may be utilized to create a well-fitting eyeglass frame for the subject.

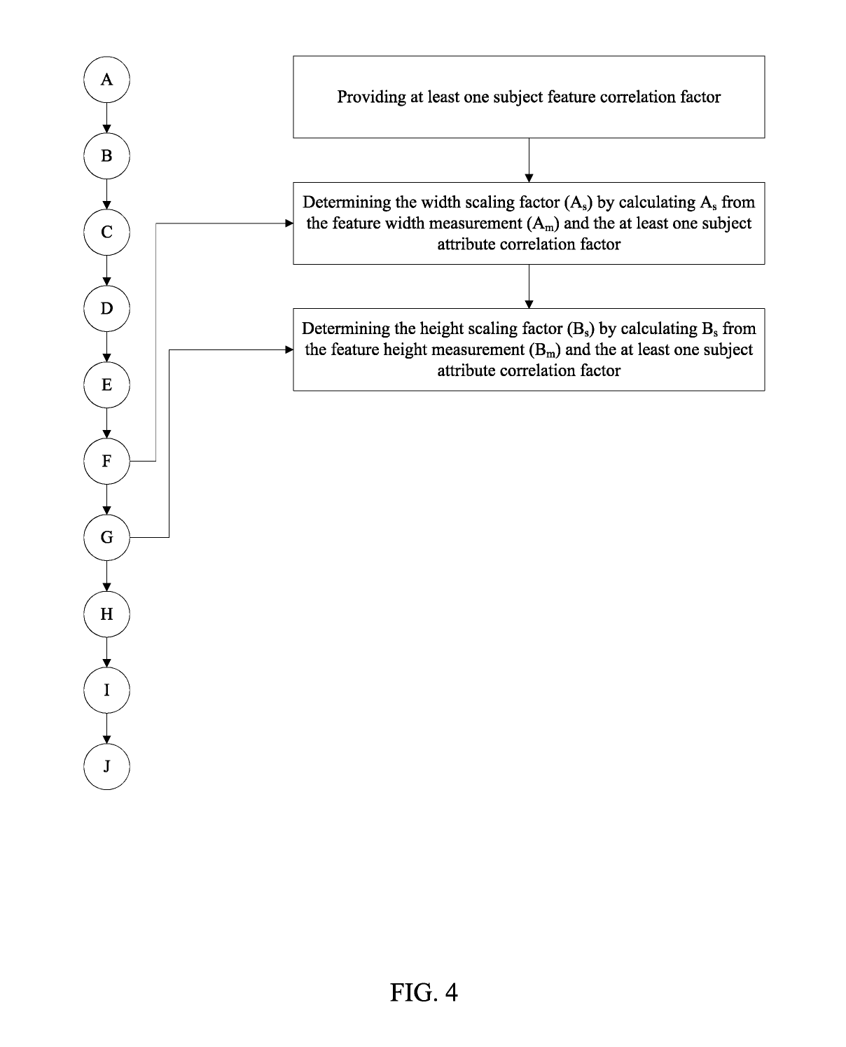

In order to scale the height and width measurements in the present invention, at least one subject feature correlation factor must be provided, as described in FIG. 4. The width scaling factor (Aₛ) is determined by calculating Aₛ from the feature width measurement (Aₘ) and the at least one subject attribute correlation factor. Similarly, the height scaling factor (Bₛ) is determined by calculating Bₛ from the feature width measurement (Bₘ) and the at least one subject attribute correlation factor. Further detail on calculating Aₛ and Bₛ will be provided further on in the present disclosure.

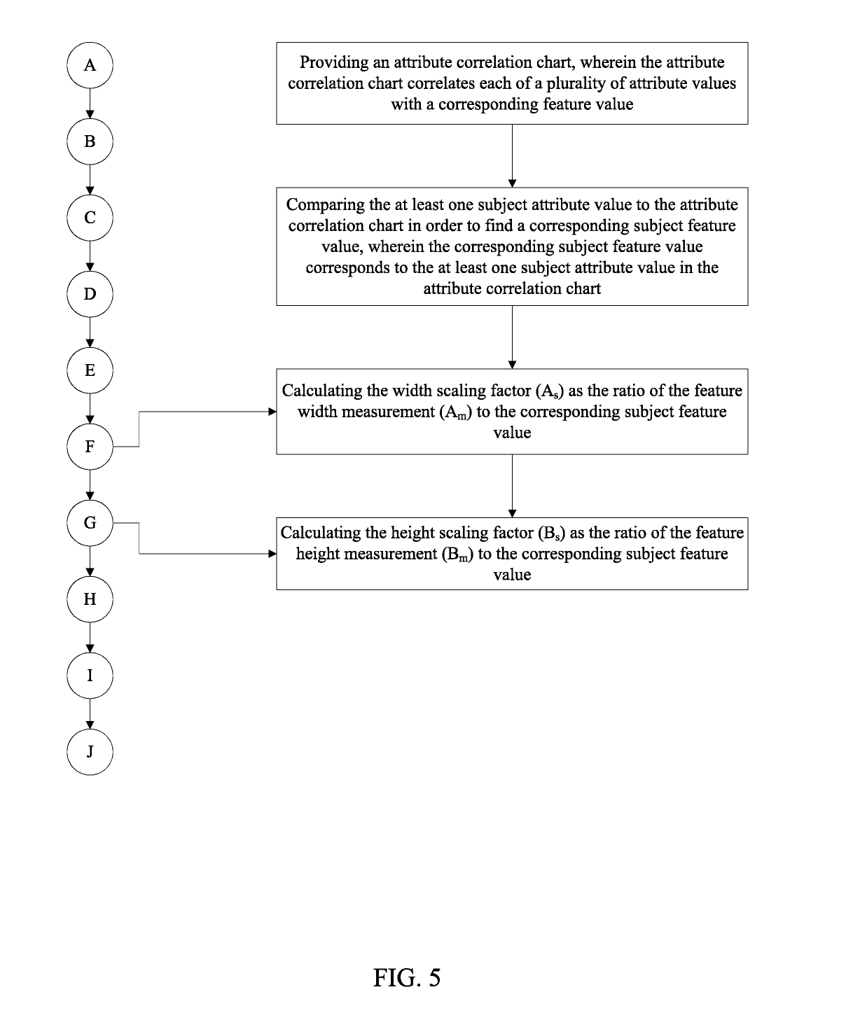

In the preferred embodiment of the present invention, an attribute correlation chart is provided, as described in FIG. 5. The attribute correlation chart correlates each of a plurality of attribute values with a corresponding feature value. The attribute correlation chart may additionally or alternatively take the form of one or more mathematical formulas that outputs a corresponding feature value given the inputs of the at least one subject attribute value. In general, the attribute correlation chart represents any means of using the at least one subject attribute value to identify one or more corresponding feature values. The present invention should be understood to not be limited to using a chart for the correlation, and alternate embodiments may utilize any other means which accomplishes the same purpose.

The at least one subject attribute value is compared to the attribute correlation chart in order to find a corresponding subject feature value, wherein the corresponding subject feature value corresponds to the at least one subject attribute value in the attribute correlation chart. The width scaling factor (Aₛ) is then calculated as the ratio of the feature width measurement (Aₘ) to the corresponding subject feature value, and similarly the height scaling factor (Bₛ) is calculated as the ratio of the feature height measurement (Bₘ) to the corresponding subject feature value.

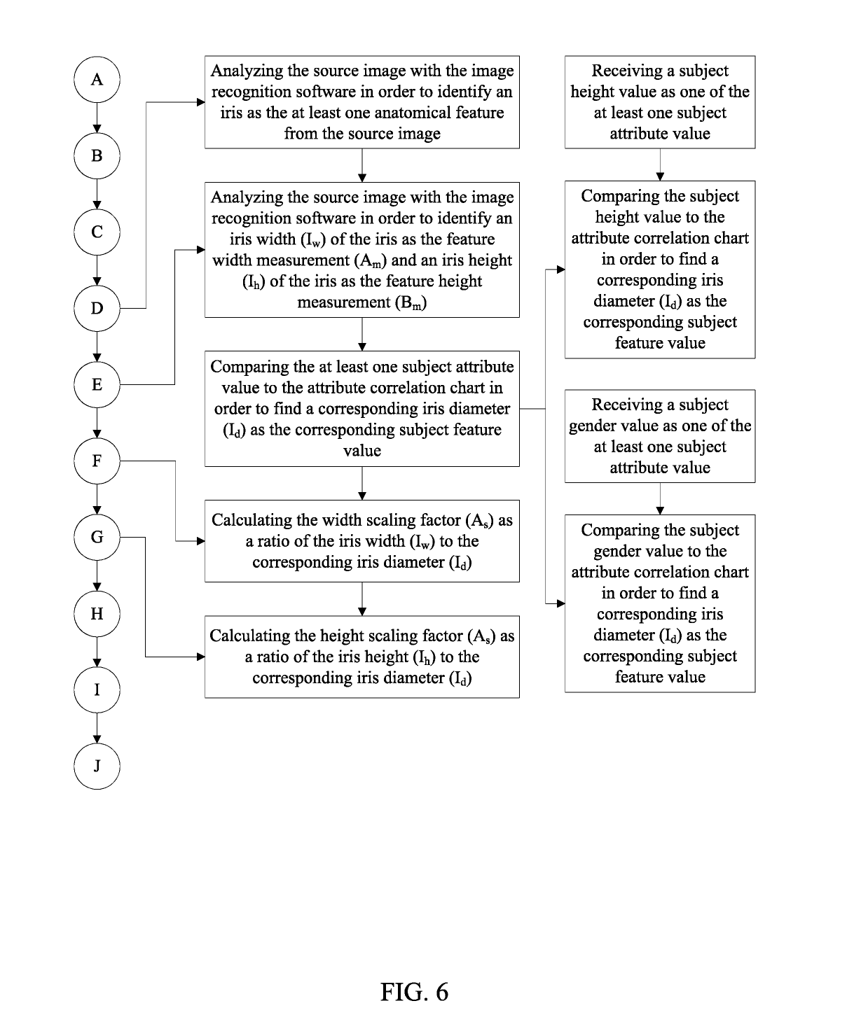

Referring to FIG. 6, in the preferred embodiment of the present invention, the at least one subject attribute value comprises a subject height value and a subject gender value, and the corresponding feature value is the average iris diameter corresponding to the height and gender values. Thus, in the preferred embodiment, the source image is initially analyzed with the image recognition software in order to identify an iris as the at least one anatomical feature from the source image. The source image is further analyzed with the image recognition software in order to identify an iris width (I_{w}) of the iris as the feature width measurement (Aₘ) and an iris height (I_{b}) of the iris as the feature height measurement (Bₘ).

The at least one subject attribute value is compared to the attribute correlation chart in order to find a corresponding iris diameter (I_{d}) as the corresponding subject feature value. The width scaling factor (Aₛ) is then calculated as a ratio of the iris width (I_{w}) to the corresponding iris diameter (I_{d}), and the height scaling factor is calculated as a ratio of the iris height (I_{b}) to the corresponding iris diameter (I_{d}).

Furthermore, in the preferred embodiment, a subject height value is received as one of the at least one subject attribute value. The subject height value is compared to the attribute correlation chart in order to find the corresponding iris diameter (I_{d}) as the corresponding subject feature value. Alternatively or additionally, a subject gender value is received as one of the at least one subject attribute value, and the subject gender value is compared to the attribute correlation chart in order to find the corresponding iris diameter (I_{d}) as the corresponding subject feature value. It is contemplated that in one embodiment, only the subject height value may be utilized to find I_{d}, in one embodiment only the subject gender value may be utilized to find I_{d}, in one embodiment both the subject height value and the subject gender value may be utilized to find I_{d}, and in other alternative embodiments other subject attribute values may be additionally or alternatively utilized.

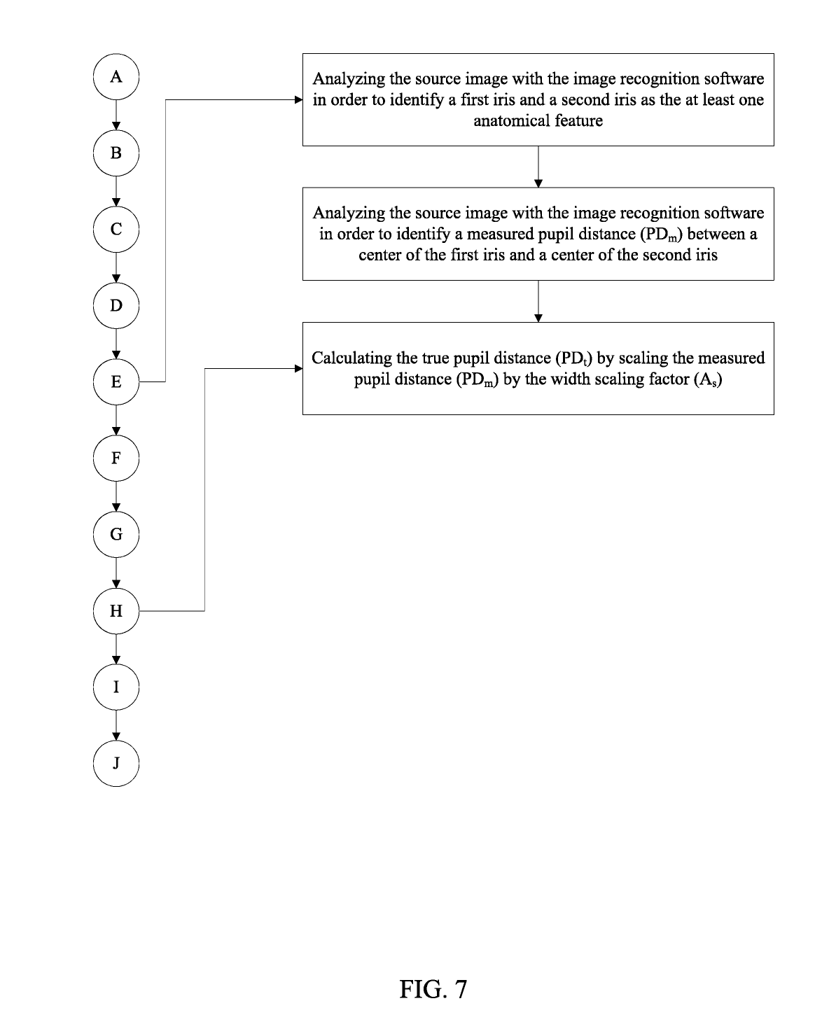

In the preferred embodiment, the source image is analyzed with the image recognition software in order to identify a first iris and a second iris as the at least one anatomical feature, as described in FIG. 7. The source image is further analyzed with the image recognition software in order to identify a measured pupil distance (PDₘ) between a center of the first iris and a center of the second iris. The true pupil distance (PDₜ) is then calculated by scaling the measured pupil distance (PDₘ) by the width scaling factor (Aₛ).

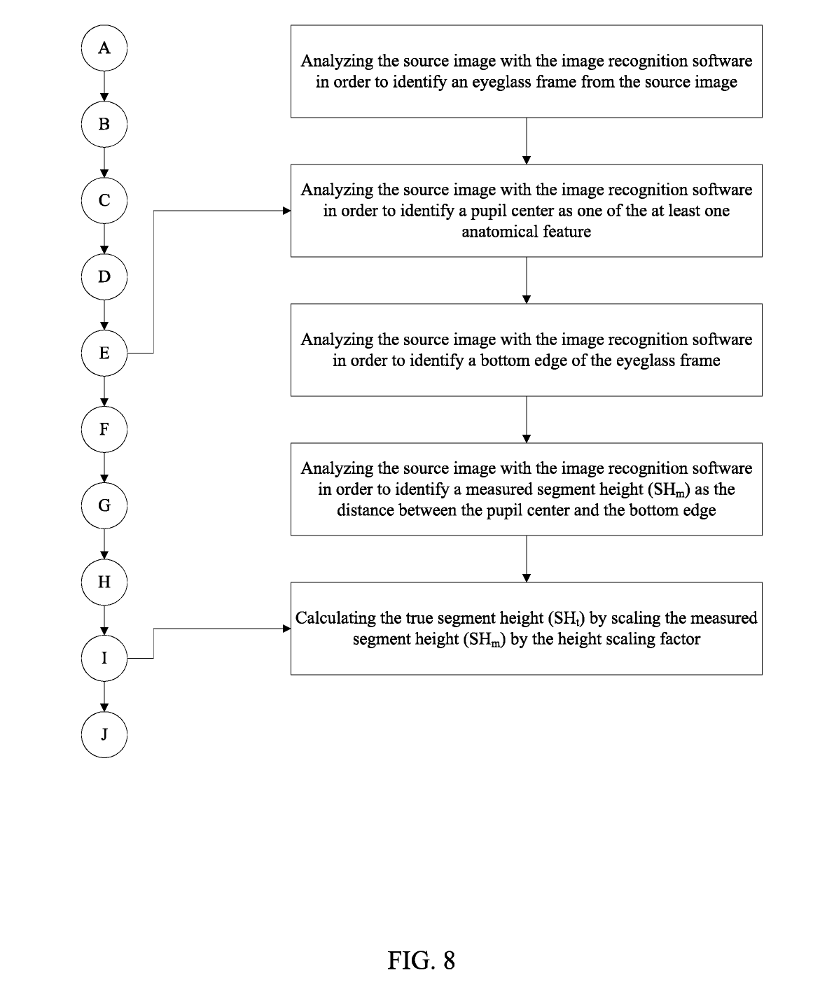

Referring to FIG. 8, in one embodiment, the subject is wearing a pair of eyeglass frames in the source image. The source image is analyzed with the image recognition software in order to identify an eyeglass frame from the source image, in addition to identifying a pupil center as one of the at least one anatomical feature, and a bottom edge of the eyeglass frame. The source image is further analyzed with the image recognition software in order to identify a measured segment height (SHₘ) as the distance between the pupil center and the bottom edge, and the true segment height (SHₑ) is calculated by scaling the measured segment height (SHₘ) by the height scaling factor.

Although the invention has been explained in relation to its preferred embodiment, it is to be understood that many other possible modifications and variations can be made without departing from the spirit and scope of the invention as hereinafter claimed.

What is claimed is:

1. A method of determining eyeglass fitting measurements from an image for a vision treatment by executing computer-executable instructions stored on a non-transitory computer-readable medium comprises the steps of:
(A) providing image recognition software;
(B) receiving a source image through a user interface;
(C) receiving at least one subject attribute value through the user interface;
(D) analyzing the source image with the image recognition software in order to identify at least one anatomical feature from the source image;
(E) analyzing the source image with the image recognition software in order to identify a feature width measurement (Aₘ) and a feature height measurement (Bₘ) from the at least one anatomical feature;
(F) determining a width scaling factor (Aₛ) from the at least one subject attribute value and the feature width measurement (Aₘ);
(G) determining a height scaling factor (Bₛ) from the at least one subject attribute value and the feature height measurement (Bₘ);
(H) calculating a true pupil distance (PDₜ) from the feature width measurement (Aₘ) and the width scaling factor (Aₛ);
(I) calculating a true segment height (SHₜ) from the feature height measurement (Bₘ) and the height scaling factor (Bₛ); and
(J) displaying the true pupil distance (PDₜ) and the true segment height (SHₜ) on a display device.

2. The method of determining eyeglass fitting measurements from an image for a vision treatment by executing computer-executable instructions stored on a non-transitory computer-readable medium as claimed in claim 1 comprises the steps of:
providing at least one subject feature correlation factor;
determining the width scaling factor (Aₛ) by calculating Aₛ from the feature width measurement (Aₘ) and the at least one subject attribute correlation factor; and
determining the height scaling factor (Bₛ) by calculating Bₛ from the feature height measurement (Bₘ) and the at least one subject attribute correlation factor.

3. The method of determining eyeglass fitting measurements from an image for a vision treatment by executing computer-executable instructions stored on a non-transitory computer-readable medium as claimed in claim 1 comprises the steps of:
providing an attribute correlation chart, wherein the attribute correlation chart correlates each of a plurality of attribute values with a corresponding feature value;
comparing the at least one subject attribute value to the attribute correlation chart in order to find a corresponding subject feature value, wherein the corresponding subject feature value corresponds to the at least one subject attribute value in the attribute correlation chart;
calculating the width scaling factor (Aₛ) as the ratio of the feature width measurement (Aₘ) to the corresponding subject feature value; and
calculating the height scaling factor (Bₛ) as the ratio of the feature height measurement (Bₘ) to the corresponding subject feature value.

4. The method of determining eyeglass fitting measurements from an image for a vision treatment by executing computer-executable instructions stored on a non-transitory computer-readable medium as claimed in claim 3 comprises the steps of:
analyzing the source image with the image recognition software in order to identify an iris as the at least one anatomical feature from the source image;
analyzing the source image with the image recognition software in order to identify an iris width (I_{w}) of the iris as the feature width measurement (Aₘ) and an iris height (I_{b}) of the iris as the feature height measurement (Bₘ);
comparing the at least one subject attribute value to the attribute correlation chart in order to find a corresponding iris diameter (I_{d}) as the corresponding subject feature value;
calculating the width scaling factor (Aₛ) as a ratio of the iris width (I_{w}) to the corresponding iris diameter (I_{d}); and
calculating the height scaling factor (Aₛ) as a ratio of the iris height (I_{b}) to the corresponding iris diameter (I_{d}).

5. The method of determining eyeglass fitting measurements from an image for a vision treatment by executing computer-executable instructions stored on a non-transitory computer-readable medium as claimed in claim 3 comprises the steps of:
receiving a subject height value as one of the at least one subject attribute value; and
comparing the subject height value to the attribute correlation chart in order to find a corresponding iris diameter (I_{d}) as the corresponding subject feature value.

6. The method of determining eyeglass fitting measurements from an image for a vision treatment by executing computer-executable instructions stored on a non-transitory computer-readable medium as claimed in claim 3 comprises the steps of:
receiving a subject gender value as one of the at least one subject attribute value; and
comparing the subject gender value to the attribute correlation chart in order to find a corresponding iris diameter (I_{d}) as the corresponding subject feature value.

7. The method of determining eyeglass fitting measurements from an image for a vision treatment by executing computer-executable instructions stored on a non-transitory computer-readable medium as claimed in claim 1 comprises the steps of:
analyzing the source image with the image recognition software in order to identify a first iris and a second iris as the at least one anatomical feature;
analyzing the source image with the image recognition software in order to identify a measured pupil distance (PDₘ) between a center of the first iris and a center of the second iris; and
calculating the true pupil distance (PDₜ) by scaling the measured pupil distance (PDₘ) by the width scaling factor (Aₛ).

8. The method of determining eyeglass fitting measurements from an image for a vision treatment by executing computer-executable instructions stored on a non-transitory computer-readable medium as claimed in claim 1 comprises the steps of:
analyzing the source image with the image recognition software in order to identify an eyeglass frame from the source image;
analyzing the source image with the image recognition software in order to identify a pupil center as one of the at least one anatomical feature;
analyzing the source image with the image recognition software in order to identify a bottom edge of the eyeglass frame;
analyzing the source image with the image recognition software in order to identify a measured segment height (SHₘ) as the distance between the pupil center and the bottom edge; and
calculating the true segment height (SHₜ) by scaling the measured segment height (SHₘ) by the height scaling factor.
